(19) United States
(12) Patent Application Publication
(10) Pub. No.: US 2019/0142541 A1
(43) Pub. Date: May 16, 2019

(54) SURGICAL DRAPE WITH TRANSPARENT SHEET

(71) Applicant: 3M INNOVATIVE PROPERTIES COMPANY, SAINT PAUL, MN (US)

(72) Inventors: Karen NEIS, ANDOVER, MN (US); Benjamin B. GULSVIG, WOODBURY, MN (US); Joanna Esther Z. SCHNEIDER, STILLWATER, MN (US)

(73) Assignee: 3M INNOVATIVE PROPERTIES COMPANY, SAINT PAUL, MN (US)

(21) Appl. No.: 16/098,545

(22) PCT Filed: May 1, 2017
(86) PCT No.: PCT/US17/30346

§ 371 Date: Nov. 2, 2018

Related U.S. Application Data
(60) Provisional application No. 62/335,441, filed on May 12, 2016.

(51) Int. Cl. A61B 46/20; A61B 90/40; A61B 46/00

(52) U.S. Cl. CPC A61B 46/20; A61B 2017/00902; A61B 46/40; A61B 90/40

(57) ABSTRACT

A surgical drape comprising a flexible opaque sheet, a flexible transparent sheet disposed adjacent the opaque sheet to permit the patient to view the surgical procedure therethrough, the transparent sheet being sealed along a substantial portion of its bottom edge to the opaque sheet, one or more fasteners configured to dispose the opaque sheet adjacent the transparent sheet, wherein the opaque sheet is configured to selectively cover and uncover the transparent sheet, one or more engagement mechanism disposed at a top edge of the transparent sheet capable of engaging with a support structure, and the transparent sheet configured for being selectively covered and uncovered during the surgical procedure to respectively prevent and permit the patient to view progress of the surgical procedure without affecting surgical access to the surgical field, the top edge of the transparent sheet extending beyond a portion of the top edge of the opaque sheet.

### BACKGROUND

This invention relates to a surgical drape, and more particularly to a surgical drape for Caesarian section procedures, the drape having a coverable transparent sheet oriented to allow a patient and her husband or other companion to selectively view the birth of their child and other aspects of the procedure.

When preparing a patient for surgery, the area of the incision (the surgical area or surgical field) is sterilized to help prevent infection. Sterilized surgical drapes are then typically placed about the sterilized surgical area. The surgical drapes provide a barrier which helps to isolate the sterilized surgical area from non-sterilized surroundings. During surgical operations such as Caesarian section procedures, the surgical drape also serves to protect the patient, and any nearby observers (e.g., the father or other family members) from fluids released during the procedure.

These conventional surgical drapes are designed and deployed to provide the surgical team with an unencumbered view of the surgical area. Little or no consideration, however, is typically given to enabling the patient or family member to view the procedure. Rather, for procedures in which the patient is conscious, conventional drapes are generally used to intentionally block the patient's view of the surgical area. For example, a Caesarian section drape customarily forms a relatively large vertical overhang, which effectively forms an opaque wall between the patient's face and the surgical field. This wall disadvantageously tends to obstruct the parents' view of the birth.

In order to allow the parents to view the birth, the drape must be lowered or removed, potentially exposing the surgical field to infection. Lowering or removing the surgical drape also potentially exposes the parents to blood and other fluid from the operation, while also permitting the parents to view other aspects of the surgery (e.g., the surgical incision, stitching, etc.), that they may not wish to see.

A need therefore exists for a sterilizable surgical drape for surgical procedures, such as Caesarian sections, which provides the desired barrier functionality, while selectively enabling the patient (and family members) to view the birth of their child as well as some aspects of the surgery, and blocking the view of other aspects of the procedure.

### SUMMARY

One aspect of the invention includes a surgical drape for use in a surgical procedure on a patient, comprising: a flexible opaque sheet; a flexible transparent sheet disposed adjacent the opaque sheet to permit the patient to view at least a portion of the surgical procedure therethrough; the transparent sheet being sealed along a substantial portion of its bottom edge to said opaque sheet; one or more fasteners configured to dispose the opaque sheet adjacent said transparent sheet, wherein the opaque sheet is configured to selectively cover and uncover the transparent sheet; one or more engagement mechanism disposed at a top edge of the transparent sheet capable of engaging with a support structure; and the transparent sheet configured for being selectively covered and uncovered during the surgical procedure to respectively prevent and permit the patient to view progress of the surgical procedure without affecting surgical access to the surgical field; the top edge of the transparent sheet extending beyond at least a portion of the top edge of the opaque sheet.

### BRIEF DESCRIPTION OF THE DRAWINGS

The above and other features and advantages of this invention will be more readily apparent from a reading of the following detailed description of various aspects of the invention taken in conjunction with the accompanying drawings, in which:

FIG. 1 is a schematic front view of a first embodiment of a surgical drape of the present invention;

FIG. 2 is a cross-sectional view along line A-A of the surgical drape of FIG. 1;

FIG. 3 is a schematic front view of a second embodiment of a surgical drape of the present invention;

FIG. 4 is a cross-sectional view along line B-B of the surgical drape of FIG. 3;

FIG. 5 is a perspective view of a surgical drape of the present invention in use with the opaque sheet attached to the transparent sheet; and

FIG. 6 is a perspective view of a surgical drape of the present invention in use with the opaque sheet detached to the transparent sheet.

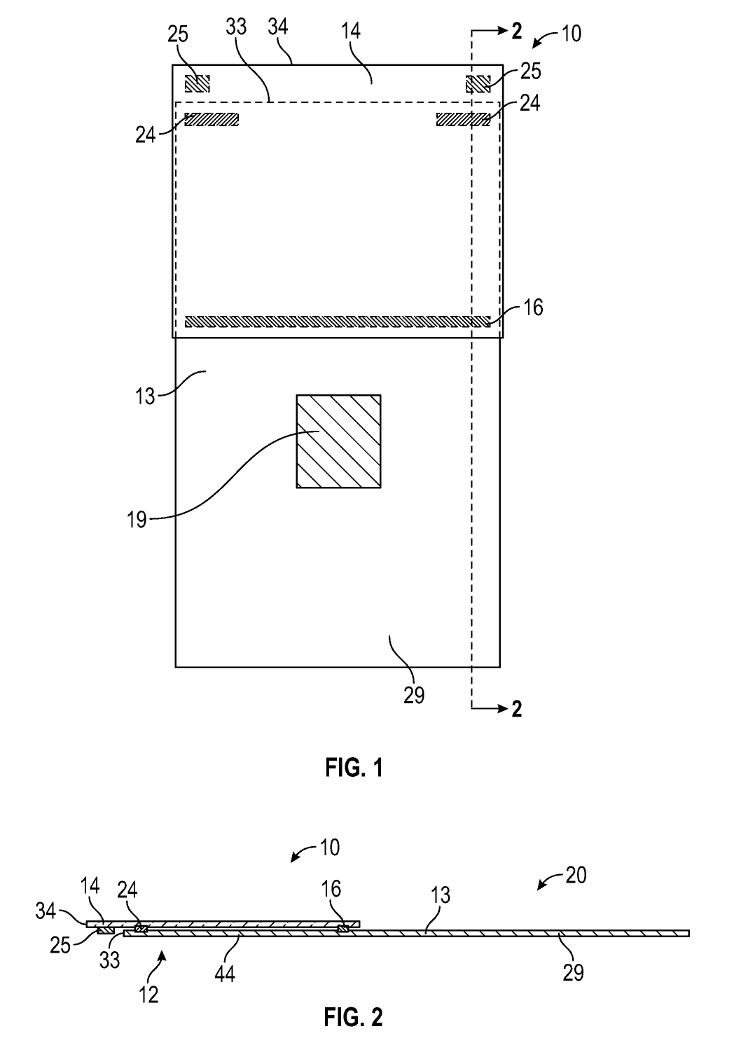

### DETAILED DESCRIPTION

In the following detailed description, reference is made to the accompanying drawings that form a part hereof, and in which is shown by way of illustration, specific embodiments in which the invention may be practiced. These embodiments are described in sufficient detail to enable those skilled in the art to practice the invention, and it is to be understood that other embodiments may be utilized. It is also to be understood that structural, procedural and system changes may be made without departing from the spirit and scope of the present invention. The following detailed description is, therefore, not to be taken in a limiting sense, and the scope of the present invention is defined by the appended claims and their equivalents. For clarity of exposition, like features shown in the accompanying drawings are indicated with like reference numerals and similar features are indicated with similar reference numerals.

Where used in this disclosure, the term “patient side” or “front side” refers to a side of the surgical drape facing the head of the patient. “Surgical side” or “rear side” refers to a side of the surgical drape opposite the patient side, i.e., which does not face the head of the patient. In addition, it should be noted that the mother during a Caesarian section may have a loved one/family member present. For convenience, this specification refers collectively to the mother and loved one/family member as the “parents”.

An aspect of the invention was the realization by the instant inventor that during Caesarian section procedures, parents often wish to view the birth of the child, but that such viewing was thwarted or rendered substantially impossible by conventional surgical drapes. As discussed above, these conventional drapes are typically deployed in a manner which blocks the parents' view. As also discussed, lowering the drape to allow the parents to see the birth has drawbacks including potentially exposing the surgical area to infection, while also exposing the parents to aspects of the surgery from which they may wish to be shielded. The inventor determined that a sterilizable clear plastic sheet disposed on the drape, would not compromise the sterility of the surgical area (or the ability to sterilize the drape for re-use), while allowing the parents to view the birth. The inventor also determined that a sterilizable, selectively deployable opaque sheet integrated into the drape, may be used to enable the parents to view the birth, and to shield the parents from other aspects of the procedure.

For example, during the preliminary steps of a typical Caesarian section procedure, the opaque sheet may be maintained adjacent the transparent sheet to block the parents' view. The surgical team may then remove the opaque sheet to permit the parents to view the birth, while protecting both the parents and the sterility of the surgical field. The opaque sheet may be replaced for the remainder of the procedure.

Turning now the Figures, embodiments of the present invention will be discussed in detail. Drape 10 includes an opaque sheet material 13 releasably attached to a transparent sheet 14. Transparent sheet 14 is sized and shaped to provide the patient undergoing the procedure with a relatively unobstructed view of the surgical field when the drape is suspended over the patient in a conventional manner. A patient may thus observe a surgical procedure through transparent sheet 14 along patient viewing axis 18, when the opaque sheet 13 is lowered.

The opaque sheet material 13 may be substantially any material commonly used for surgical drapes Moreover, although it is contemplated that the embodiments of drape 10 shown and described herein may be disposable, in particular embodiments, drape 10 is sterilizable (e.g., gas or gamma sterilizable), to facilitate re-use.

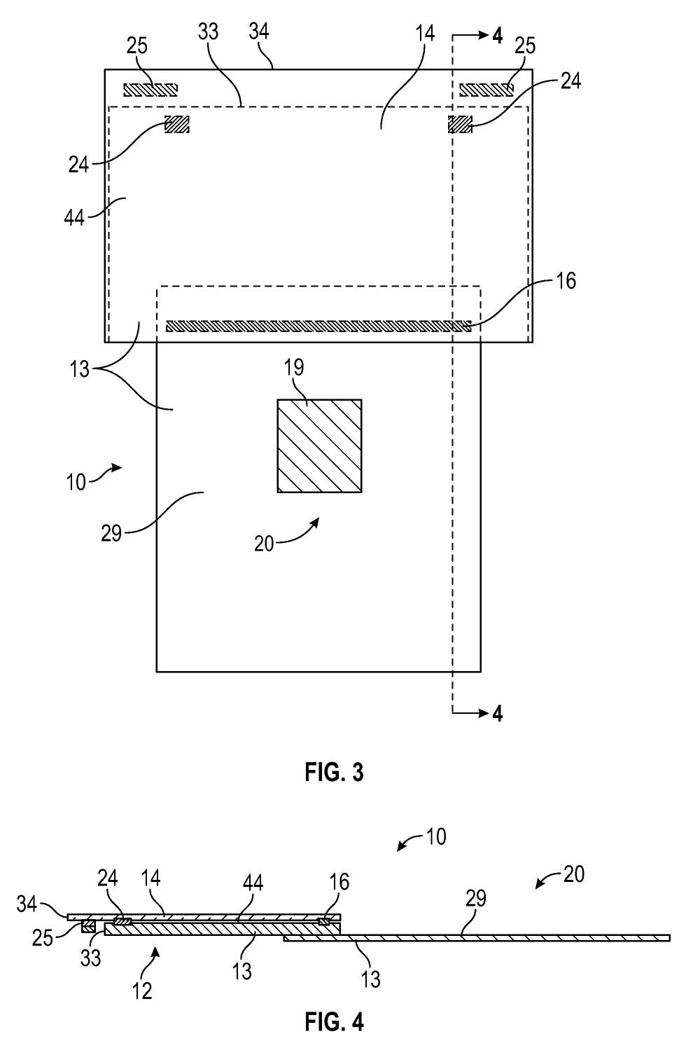

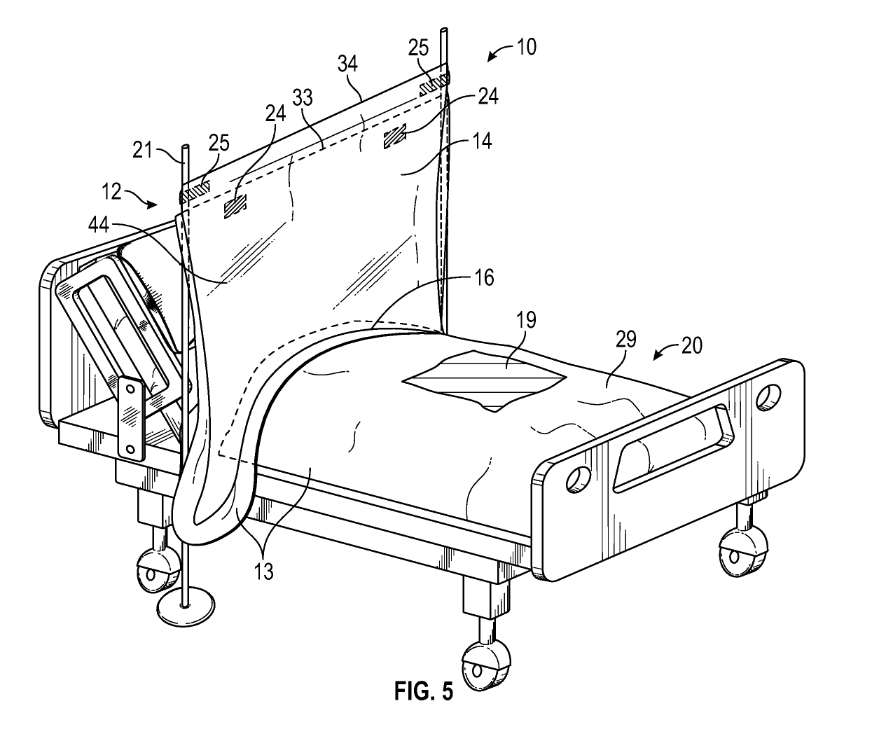

Examples of sheet material that may be suitable for various embodiments of opaque sheet 13 of drape 10 include woven or non-woven absorbent laminates, barrier composites, and barrier fabrics, such as those available from Precision Fabrics Group, Inc. (Vinton, Va.). SMS non-woven material (spunbond-meltblown-spunbond) material, and/or 3M™ Ioban™ 2 antimicrobial drape material available from 3M (St. Paul, Minn.) may also be used in some applications. In at least one embodiment, the drape has two distinct portions: a surgical portion 29 and an anaesthesia portion 44. The opaque material preferably has two distinct sides: one side is impervious to liquids and the other is absorbent. The drape may be made of a single sheet of opaque material, e.g., as shown in FIG. 1, or may be made of two or more sheets of opaque material joined together, e.g., as shown in FIG. 3. In at least one embodiment of the drape, the side of the surgical portion 29 of the drape facing the patient is impervious to liquid and the side of the anaesthesia portion 44 of the drape facing the patient is absorbent.

Transparent sheet 14 may be fabricated from any one or more materials, which are substantially transparent and flexible. Examples of potentially suitable materials include polymeric (plastic) sheet materials, e.g., polyethylene (PE), polyvinylchloride (PVC), polypropylene (PP), and combinations or mixtures thereof.

As mentioned above, in particular embodiments, the transparent sheet is fabricated from a material that is capable of being sterilized by gas or gamma-sterilization techniques commonly used in hospitals and other medical facilities. Examples of gas-sterilization techniques usable with the various embodiments discussed herein, include those using Ethylene Oxide (EtO) gas, e.g., those known as gas diffusion sterilization techniques.

Transparent sheet 14 is secured to the opaque sheet material 13 along its bottom edge with a seal 16 that preferably meets or exceeds the barrier performance of the drape material. For example, a suitable seal 16 may be provided by heat fusion, ultrasonic welding, chemical adhesives, and/or combinations thereof, depending on the particular materials used for sheet 13 and transparent sheet 14. Transparent sheet 14 may be disposed along an edge of the drape, so that a portion of the periphery of transparent sheet 14 forms an outer edge of drape 10, and thus is not secured to sheet 13.

Transparent sheet 14 is additionally releasably attached to opaque sheet 13 near their top edges by fasteners 24. The fasteners 24 may be reusable or single use. Examples of reusable fasteners include hook and loop closures. Examples of single use fasteners include perforations. If single use fasteners are used, the opaque and transparent sheets may be reattached by other means such as a clip.

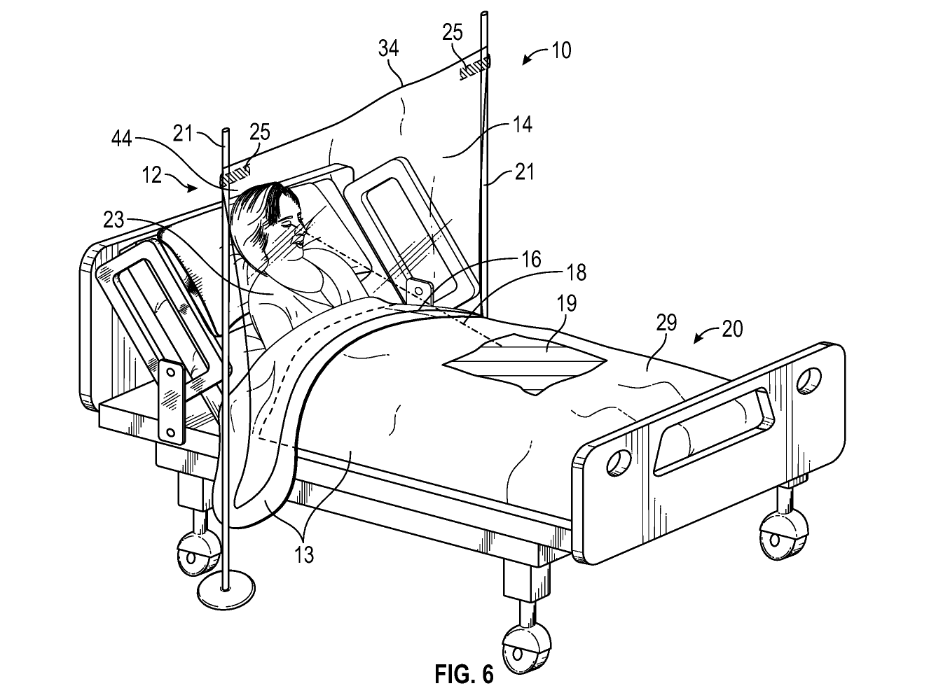

An advantage of transparent sheet 14 of the present invention is that it is relatively large thereby ensuring an unobstructed patient view in a wide range of applications. The transparent sheet is typically attached to a support structure 21 such as a stand or pole on each side of the patient's bed by an engagement mechanism 25. The engagement mechanism 25 may be re-usable or suitable for one time use. The engagement mechanism 25 may be an adhesive, initially covered by a release liner, which is removed when the transparent sheet is attached to the support structure. In at least one embodiment, the anaesthesia portion 44 of the opaque sheet is on the patient side of the drape and the transparent sheet 14 is on the surgical side of this anaesthesia portion 44. Because the engagement mechanism 25, which is used to attach the transparent sheet 14 to a support structure 21 (which may be a conventional intravenous (I.V.) pole), is on the patient side of the transparent sheet, the opaque sheet must be appropriately configured to allow the engagement mechanism 25 to engage with the support structure. This may be accomplished by the top edge 33 of the opaque sheet 13 being lower than the top edge 34 of the transparent sheet 14 such that the portion of the transparent sheet having the engagement mechanism 25 is exposed. In some embodiments, as illustrated in FIG. 5, the entire top edge 33 of the opaque sheet may be lower than the top edge of the transparent sheet 34. In other embodiments, only the portion of the opaque sheet that would cover the engagement mechanism 25 need be lower.

These embodiments thus represent an improvement over currently available surgical drapes, which as mentioned above, are configured primarily to help maintain a sterile surgical field at the area of the incision, and to protect the patient/family members from fluids, but which are not configured to allow the patient to selectively view aspects of the surgical procedure.

In FIG. 5, the surgical side 20 of drape 10 is shown, with opaque sheet 13 substantially covering transparent sheet 14, i.e., secured in position by fasteners 24. As also shown, opaque sheet 13 is sized and shaped to substantially match the transparent sheet.

Fasteners 24 may be seen, which are disposed near the top edge of opaque sheet 13. Substantially any type of single use or re-usable fasteners may be used, including mechanical and chemical fasteners such as mechanical snaps, perforations ties, conventional hook and loop fasteners, e.g., VELCRO® fasteners (Velcro Industries B.V.), and/or adhesives such as releasable adhesives similar to that used in Post-It® notes (3M, Minneapolis, Minn.), etc. In particular embodiments, the fasteners 24 include conventional hook and loop fasteners, e.g., VELCRO® fasteners (Velcro Industries B.V.). If a single use fastener, such as perforations are used, the opaque sheet may be re-attached to transparent sheet 14 using surgical clips along the periphery of transparent sheet 14.

As shown in FIG. 6, opaque sheet 13 may be lowered to permit the patient 23 to look through transparent sheet 14, along patient viewing axis (sight line) 18, to observe the surgical area 19. In this example, opaque sheet 13 is disposed on the patient side 12 of the drape so it can be lowered without interfering with the barrier provided by the transparent sheet 14. In alternate embodiments and/or applications, however, the opaque sheet 13 may be disposed on the surgical side of the drape.

Surgical drape 10 may be used in conjunction with other articles suitable for surgical procedures. It may be combined in a kit with such other articles, such as incise drapes.

Although embodiments of the invention are particularly suitable for Caesarian section procedures, they are not so limited. Rather, embodiments of the invention may be used with substantially any surgical procedure in which a patient is conscious and may wish to view some aspects of the surgery, and/or may be expected to answer questions during the surgery. Examples of such other surgical procedures may include hernia repairs, appendectomies, and/or orthopedic procedures.

It should be understood that any of the features described with respect to one of the embodiments described herein may be similarly applied to any of the other embodiments described herein without departing from the scope of the present invention.

In the preceding specification, the invention has been described with reference to specific exemplary embodiments for the purposes of illustration and description. It is not intended to be exhaustive or to limit the invention to the precise form disclosed. Many modifications and variations are possible in light of this disclosure. It is intended that the scope of the invention be limited not by this detailed description, but rather by the claims appended hereto.

What is claimed is:

1. A surgical drape for use in a surgical procedure on a patient, comprising:
a flexible opaque sheet;
a flexible transparent sheet disposed adjacent said opaque sheet to permit the patient to view at least a portion of the surgical procedure therethrough;
said transparent sheet being sealed along a substantial portion of its bottom edge to said opaque sheet;
one or more fasteners configured to dispose said opaque sheet adjacent said transparent sheet, wherein said opaque sheet is configured to selectively cover and uncover said transparent sheet;
one or more engagement mechanism disposed at a top edge of the transparent sheet capable of engaging with a support structure; and
said transparent sheet configured for being selectively covered and uncovered during the surgical procedure to respectively prevent and permit the patient to view progress of the surgical procedure without affecting surgical access to the surgical field;
said top edge of said transparent sheet extending beyond at least a portion of the top edge of said opaque sheet.

2. The surgical drape of claim 1 wherein the transparent sheet is on the surgical side of the drape.

3. The surgical drape of claim 1 wherein the engagement mechanism is an adhesive applied to the transparent sheet.

4. The surgical drape of claim 1 wherein the engagement mechanism is on the portion of the transparent sheet that extends beyond the top edge of the opaque sheet.

5. The surgical drape of claim 1 wherein the opaque sheet has first and second sides, the first side comprising an absorbent material and the second side comprising a liquid-impervious polymer film.

6. The surgical drape of claim 5 wherein the absorbent material is a nonwoven material.

7. The surgical drape of claim 1 wherein the opaque sheet comprises a surgical portion and an anaesthesia portion wherein the side of surgical portion facing the patient is liquid impervious and the side of the anaesthesia portion facing the surgical site is liquid impervious.

8. The surgical drape of claim 7 wherein the transparent sheet is attached to the liquid impervious side of the anaesthesia portion of the opaque sheet.

9. The surgical drape of claim 1 wherein the fastener is re-usable.

10. The surgical drape of claim 1 wherein the fastener is single use.

11. The surgical drape of claim 10 wherein the fastener is a perforated tab.

12. The surgical drape of claim 1 wherein the engagement mechanism is re-usable.

13. The surgical drape of claim 12 wherein the engagement mechanism is an adhesive.

14. A kit comprising:
the surgical drape of claim 1 and an incise drape.

15. The kit of claim 14 wherein the Incise drape comprises an antimicrobial agent.
